(19) United States
(12) Patent Application Publication
(10) Pub. No.: US 2026/0166279 A1
(43) Pub. Date: Jun. 18, 2026

(54) CATHETER PULL RING WITH ENHANCED ANCHORING CAPABILITIES

(71) Applicant: Cirtec Medical Corporation, Brooklyn Park, MN (US)

(72) Inventors: Kellen James Bodell, Plymouth, MN (US); Bee Anthony Xiong, Sioux Falls, SD (US); Jesse Francis Meyer, Albertville, MN (US)

(21) Appl. No.: 19/421,978

(22) Filed: Dec. 16, 2025

Related U.S. Application Data
(60) Provisional application No. 63/735,115, filed on Dec. 17, 2024.

(51) Int. Cl. A61M 25/01; A61M 25/00

(52) U.S. Cl. CPC A61M 25/0147; A61M 25/0009

(57) ABSTRACT

A catheter pull ring has a body that is folded into a cylindrical shape. An opening is implemented in the body. The catheter pull ring includes a hard-stop mechanism that is configured to be inserted into the opening when the body is folded into the cylindrical shape. The hard-stop mechanism is configured to engage with a portion of the body around the opening, such that the body that has been folded into the cylindrical shape is prevented from expanding or shrinking a circumference thereof beyond a predefined threshold. The hard-stop mechanism comprises a T-shaped bar or a tab with a jagged edge.

### PRIORITY DATA

The present application is a utility U.S. Patent application of provisional U.S. patent application No. 63/735,115, filed on Dec. 17, 2024, entitled “Catheter ring with enhanced anchoring capabilities”, the disclosure of which is hereby incorporated by reference in its entirety.

### BACKGROUND

Catheters are medical devices that can be inserted into a human body to facilitate minimally invasive surgery. Catheter pull rings may be fitted within the distal tip of a catheter shaft, and may be used to obtain precise control of a catheter, such as the deflection of the distal tip of the catheter in a desired direction. However, conventional catheter pull rings may have drawbacks. For example, conventional catheter pull rings lack adjustability in terms of size (e.g., diameter or circumference), which may in turn increase manufacturing lead time and/or cost. Therefore, although conventional catheter pull rings have generally been adequate, they have not been satisfactory in all aspects.

### BRIEF DESCRIPTION OF THE DRAWINGS

The present disclosure is best understood from the following detailed description when read with the accompanying figures. It is emphasized that, in accordance with the standard practice in the industry, various features are not drawn to scale and are used for illustration purposes only. In fact, the dimensions of the various features may be arbitrarily increased or reduced for clarity of discussion.

FIG. 1 is a planar view of a catheter pull ring according to a first embodiment of the present disclosure.

FIGS. 2-4 are three-dimensional perspective views of the catheter pull ring according to the first embodiment of the present disclosure.

FIG. 5 is a planar view of a catheter pull ring according to a second embodiment of the present disclosure.

FIGS. 6-8 are three-dimensional perspective views of the catheter pull ring according to the second embodiment of the present disclosure.

FIG. 9 is a three-dimensional perspective view of a catheter pull ring with a pull wire attached thereto according to various aspects of the present disclosure.

FIG. 10 is a three-dimensional perspective view of a catheter assembly that includes the catheter pull ring and a catheter shaft according to various aspects of the present disclosure.

FIG. 11 is a flowchart of a method according to various aspects of the present disclosure.

### DETAILED CATHETER PULL RING DESCRIPTION

The following disclosure provides many different embodiments, or examples, for implementing different features of the disclosure. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In addition, the present disclosure may repeat reference numerals and/or letters in the various examples. This repetition is for the purpose of simplicity and clarity and does not in itself dictate a relationship between the various embodiments and/or configurations discussed. Moreover, the formation of a feature on, connected to, and/or coupled to another feature in the present disclosure that follows may include embodiments in which the features are formed in direct contact, and may also include embodiments in which additional features may be formed interposing the features, such that the features may not be in direct contact. In addition, spatially relative terms, for example, “lower,” “upper,” “horizontal,” “vertical,” “above,” “over,” “below,” “beneath,” “up,” “down,” “top,” “bottom,” etc., as well as derivatives thereof (e.g., “horizontally,” “downwardly,” “upwardly,” etc.) are used for ease of the present disclosure of one features relationship to another feature. The spatially relative terms are intended to cover different orientations of the device including the features. Still further, when a number or a range of numbers is described with “about,” “approximate,” and the like, the term is intended to encompass numbers that are within a reasonable range including the number described, such as within +/−10% of the number described or other values as understood by person skilled in the art. For example, the term “about 5 mm” encompasses the dimension range from 4.5 mm to 5.5 mm.

The present disclosure is generally related to medical devices, and more particularly, to an improved catheter pull ring design. In that regard, steerable catheters are common and allow a physician to access the correct location of a patient's anatomy. They typically comprise an anchor near the distal tip of a catheter and a mechanism to pull or push on said anchor that runs back to the proximal end of the catheter and is actuated by a handle assembly. A catheter pull ring (also referred to as a pull ring) assembly typically comprises a metal ring welded to one or more metal wires. In addition to providing mechanical rigidity, the catheter pull ring may also be designed/used as radiopaque markers to allow visualization under X-rays or other imaging techniques in some cases, which helps healthcare providers confirm the catheter's position.

However, conventional catheter pull rings are typically manufactured to have a single size (e.g., a fixed diameter or circumference). Since an ideal catheter pull ring size is supposed to be only slightly larger (e.g., larger by 0.001˜0.002 inch) than the inner layer it fits over, any given conventional catheter pull ring may be able to accommodate a very small range of catheter sizes. If a manufacturer of a catheter cannot find an off-the-shelf catheter pull ring that happens to fit within the catheter shaft well, a catheter pull ring may need to be custom-made to accommodate the catheter. This would translate into a lengthy lead time (e.g., at least 3-4 months) and/or increase manufacturing costs.

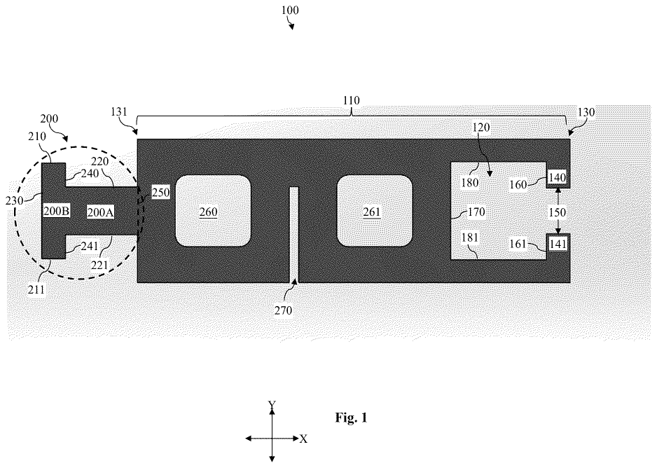

To overcome the issues discussed above, the present disclosure introduces a catheter pull ring with an adjustable size, so that the catheter pull ring—as an off-the-shelf product—can be flexibly deployed within catheters of a variety of sizes. The various aspects of the present disclosure are now discussed below with reference to FIGS. 1-11.

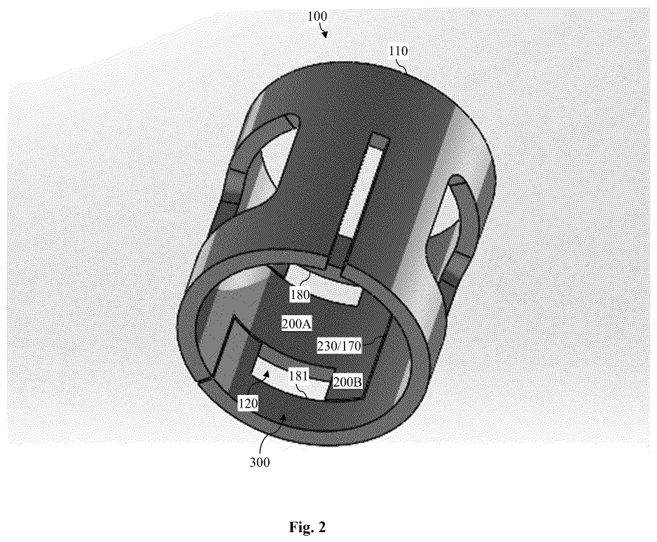

Referring now to FIGS. 1-4, a first embodiment of a catheter pull ring 100 (e.g., as an off-the-shelf device) of the present disclosure is illustrated. In more detail, FIG. 1 illustrates a planar view of the catheter pull ring 100 when the catheter pull ring 100 is fully stretched out, and FIGS. 2-4 illustrate three-dimensional perspective views of the catheter pull ring 100 when the catheter pull ring 100 is folded into a cylindrically-shaped ring, but at different configurations with different corresponding circumferences/diameters. It is understood that in some embodiments, the catheter pull ring 100 is made out of a hypotube (e.g., a hypotube comprising a stainless steel material), and it is laser cut to achieve its various patterns discussed below with reference to FIGS. 1-4. In that regard, the planar view of FIG. 1 is just for the laser programming, but the catheter pull ring 100 itself was not provided in the planar configuration.

The planar view of FIG. 1 is shown in a horizontal plane defined by an X-direction and a Y-direction perpendicular to the X-direction. As shown in FIG. 1, the catheter pull ring 100 includes a body 110 that is elongated in the X-direction and spans from one end 130 to another end 131. That is, a dimension of the body 110 in the X-direction is greater than a dimension of the body 110 in the Y-direction. The body 110 includes an opening 120 that is located at or near the end 130 of the body 110. In the illustrated embodiment, the opening 120 is defined at least in part by two protruding segments 140 and 141 that each extend in the Y-direction and are separated by a gap 150. The two protruding segments 140-141 have walls 160 and 161 that each extend in the Y-direction.

In addition to being defined by the walls 160-161, the opening 120 is further defined by a wall 170 that extends in the Y-direction and that is opposite to (and faces against) the walls 160-161, as well as walls 180 and 181 that each extend in the X-direction and are opposite to (and face against) one another. The wall 180 joins the walls 160 and 170 together, and the wall 181 joins the walls 161 and 170 together. In the embodiment illustrated in FIG. 1, the opening 120 is configured to have a rectangular shape, as each of the walls 160-161, 170, and 180-181 has a linear shape. However, it is understood that in other embodiments, the opening 120 may be configured to have non-rectangular shapes, such as an at least partially curved shape or an arbitrary shape, which may be defined by one or more non-linear walls.

The opening 120 (including the gap 150) allows a hard-stop mechanism 200 to be inserted therein. In that regard, the hard-stop mechanism 200 (shown in FIG. 1 as being covered by a dashed circle) extends away from the end 131 of the body 110 in the X-direction. The hard-stop mechanism 200 in the illustrated embodiment is shaped as a letter “T” and may therefore be interchangeably referred to as a T-shaped bar or a T-shaped tab hereinafter. In that regard, the hard-stop mechanism 200 includes a segment 200A that is directly attached to the end 131 of the body 110, as well as a segment 200B that is directly attached to the segment 200A. The segment 200A includes edges 220 and 221 that each extend in the X-direction, and the segment 200B includes edges 210 and 211 that each extend in the X-direction and edges 230 and 240-241 that each extend in the Y-direction. The segment 200B has a greater dimension in the Y-direction than the segment 200A, thereby giving the hard-stop mechanism 200 the T-shape in the planar view of FIG. 1. For example, the edge 210 of the segment 200B protrudes farther outward in the Y-direction than the edge 220 of the segment 200A, and the edge 211 of the segment 200B protrudes farther outward in the Y-direction than the edge 221 of the segment 200A. As such, the edge 230 (extending in the Y-direction) of the segment 200B has a greater dimension in the Y-direction than an imaginary interface 250 (also extending in the Y-direction) between the segment 200A and the body 110.

Note that the configuration of the hard-stop mechanism 200 shown in FIG. 1 is not intended to be limiting unless otherwise claimed. In some other embodiments, the segments 200A and 200B may be implemented such that the edge 210 protrudes beyond the edge 220, but the edge 211 does not protrude beyond the edge 221, or that the edge 211 protrudes beyond the edge 221, but the edge 210 does not protrude beyond the edge 220. Furthermore, although the segments 200A and 200B are each shaped as a rectangle in the illustrated embodiment, they may be configured to have other shapes in other embodiments, such as having an at least partially curved shape or an arbitrary shape, which may be defined by one or more non-linear edges.

The body 110 also includes reflow holes 260-261. The reflow holes 260-261 are configured to allow a reflow material to flow therethrough when the body is coupled to a catheter shaft, which may laminate the catheter pull ring 100 (including the body 110 and the hard-stop mechanism 200) within the catheter shaft. The catheter pull ring 100 may then serve as an anchor to a distal end of the catheter shaft.

The body 110 further includes a slot 270 that extends in the Y-direction. The slot 270 extends partially through the body 110 and between the reflow holes 260-261. In some embodiments, the slot 270 is configured to receive an attachment mechanism (e.g., a metal wire), which may be laser welded to the body 110 through the slot 270. A pull on the attachment mechanism may cause a deflection of the catheter in a given direction. The attachment mechanism may be actuated by a handle assembly attached to a proximal end of the catheter shaft to control a deflection of the catheter. For example, in some embodiments, a first wire and a second wire may be implemented as the attachment mechanisms to be attached to opposite sides of the catheter pull ring 100. The handle assembly may be actuated to pull on the first wire, which may cause the catheter pull ring 100 to deflect the distal end of the catheter in a first direction. The handle may also be actuated to pull on the second wire, which may cause the catheter pull ring 100 to deflect the distal end of the catheter in a second direction different from the first direction.

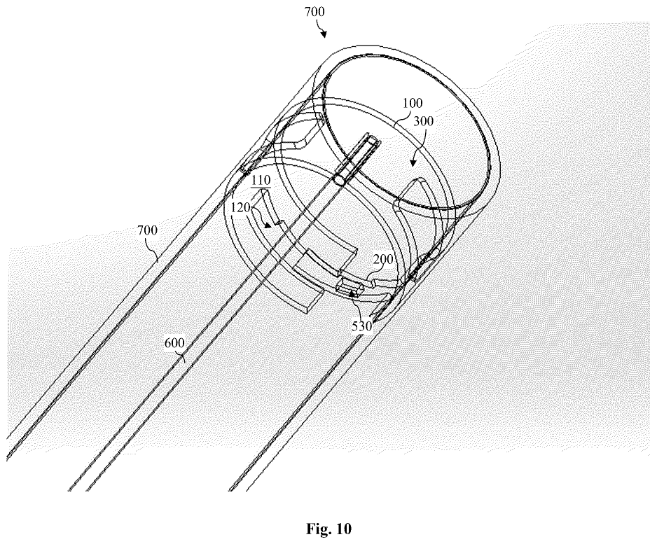

Referring now to FIG. 2, the catheter pull ring 100 is folded or bent into a cylindrical shape, such that a channel 300 is defined by the folded catheter pull ring 100. As discussed above, the catheter pull ring 100 may be made by laser cutting a stainless hypotube (which is already in a cylindrical shape) in some embodiments. As such, the channel 300 may have a circular shape in this embodiment, and as such, the channel 300 may have a circumference or a diameter that corresponds to a size of the catheter pull ring 100. A distal end of a catheter shaft (not illustrated in FIG. 2 but will be discussed below with reference to FIG. 10) can be inserted into the channel 300 to be anchored by the catheter pull ring 100.

In this ring configuration, the hard-stop mechanism 200 is inserted into the opening 120 (also see FIG. 1). Before and/or during the attachment of the catheter pull ring 100 to the catheter shaft, the hard-stop mechanism 200 may be allowed to move within the opening 120. Different positions of the hard-stop mechanism 200 within the opening 120 correspond to different sizes (e.g., in terms of circumference or diameter of the channel 300) of the catheter pull ring 100. For example, FIG. 2 illustrates the configuration where the channel 300 has a minimum circumference or diameter. This is because the segment 200B is inserted fully into the opening 120, such that the edge 230 of the segment 200B makes direct physical contact with the wall 170 of the opening 120. Since the edge 230 cannot be pushed beyond the wall 170, such a configuration prevents the catheter pull ring 100 from being shrunk any further, which in turn places a minimum value for the catheter pull ring 100 (e.g., in terms of the circumference or the diameter of the channel 300 defined by the catheter pull ring 100). Note that due to the geometric design of the hard-stop mechanism 200—the segment 200B of the hard-stop mechanism 200 being wider than the segment 200A—the insertion of the hard-stop mechanism 200 into the opening 120 still leaves gaps between the segment 200A and the walls 180 and 181.

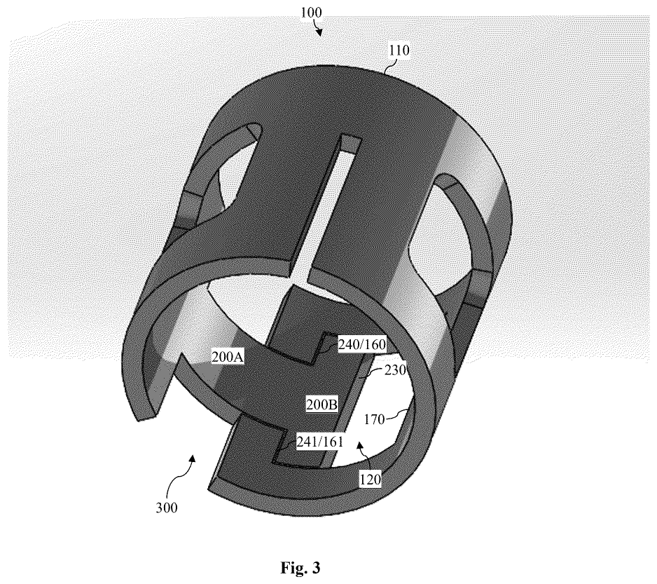

Referring now to FIG. 3, the catheter pull ring 100 is still folded or bent into a cylindrical shape, such that the channel 300 is defined by the folded catheter pull ring 100. However, the channel 300 in this configuration achieves a maximum circumference or diameter. In that regard, the hard-stop mechanism 200 is partially inserted into the opening 120, but now the edge 240 is in direct physical contact with the wall 160, and the edge 241 is in direct physical contact with the wall 161. The direct abutment between the edges 240-241 of the segment 200B of the hard-stop mechanism 200 and the walls 160-161 of the portion of the body 110 that defines the opening 120 prevents the catheter pull ring 100 from being further expanded or enlarged, which in turn places a maximum value for the catheter pull ring 100 (e.g., in terms of the circumference or the diameter of the channel 300 defined by the catheter pull ring 100). Note that in this configuration of FIG. 3, there is a gap separating the edge 230 of the hard-stop mechanism 200 from the wall 170 of the opening 120.

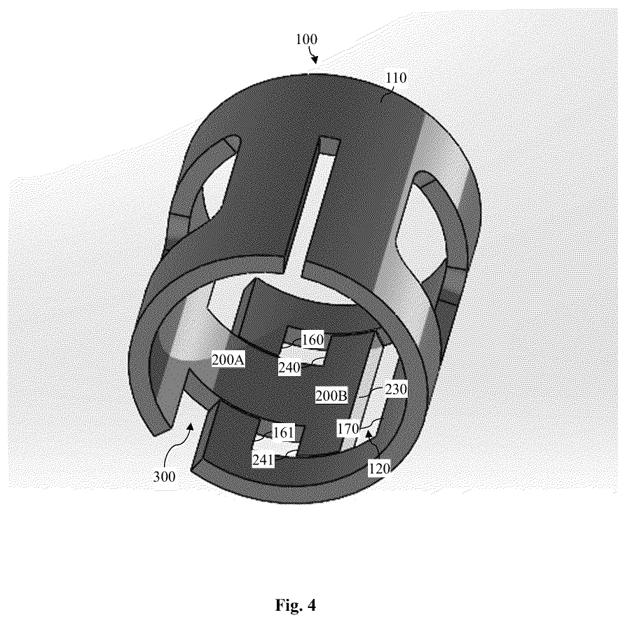

Referring now to FIG. 4, the catheter pull ring 100 is still folded or bent into a cylindrical shape, such that the channel 300 is defined by the folded catheter pull ring 100. However, the channel 300 in this configuration achieves an intermediate circumference or diameter. In that regard, the hard-stop mechanism 200 is partially inserted into the opening 120, but now the edges 240-241 are spaced apart from the walls 160-161, respectively, and the edge 230 is spaced apart from the wall 170. In this configuration, the circumference or the diameter of the channel 300 is larger than that of the configuration of FIG. 2, but smaller than that of the configuration of FIG. 3. In fact, the configuration of FIG. 4 is meant to represent any potential position of the hard-stop mechanism 200 within the opening 120. In this manner, the catheter pull ring 100 can achieve a wide range of potential ring sizes simply by moving the hard-stop mechanism 200 within the opening 120 but not directly contacting either the wall 170 or the walls 160-161.

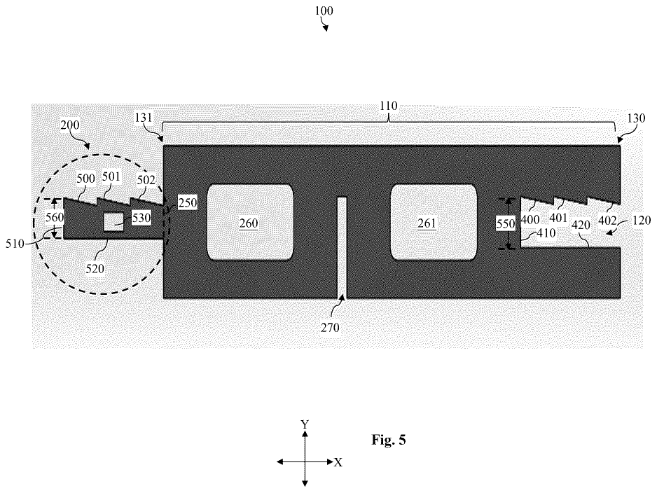

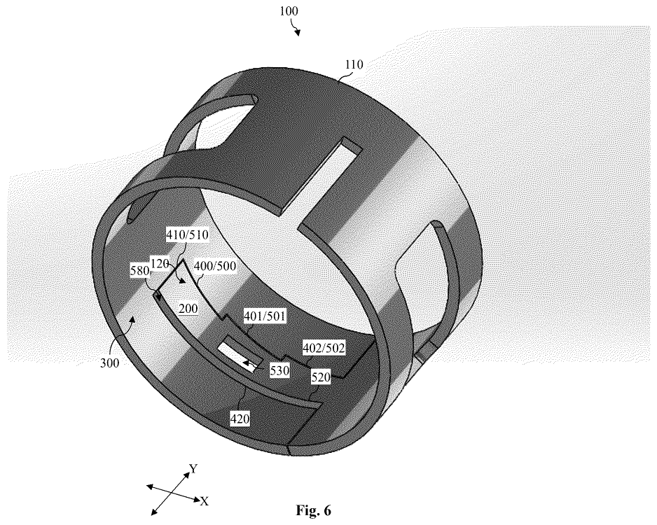

Referring now to FIGS. 5-8, a second embodiment of the catheter pull ring 100 (as an off-the-shelf device) of the present disclosure is illustrated. In more detail, FIG. 5 illustrates a planar view of the catheter pull ring 100 when the catheter pull ring 100 is fully stretched out (e.g., as an off-the-shelf device), and FIGS. 6-8 illustrate three-dimensional perspective views of the catheter pull ring 100 when the catheter pull ring 100 is folded into a cylindrically-shaped ring, but at different configurations with different corresponding circumferences/diameters. Note that for reasons of consistency and clarity, similar components appearing in the first embodiment of FIGS. 1-4 will be labeled the same in the second embodiment of FIGS. 5-8. As discussed above, the catheter pull ring 100 may be made by laser cutting a stainless hypotube in some embodiments, and thus the catheter pull ring 100 itself was not provided in the planar configuration.

Referring now to FIG. 5, the planar view shown herein also corresponds to a horizontal plane defined by the X-direction and the Y-direction perpendicular to the X-direction. The second embodiment of the catheter pull ring 100 also includes the elongated body 110 that spans from one end 130 to another end 131 and that contains the reflow holes 260-261 and the slot 270. However, the opening 120 defined by the body 110 and the hard-stop mechanism 200 attached to the end 131 of the body 110 are different than those discussed above with reference to the first embodiment of FIGS. 1-4. In more detail, the opening 120 in the second embodiment of FIGS. 5-8 is not rectangular, but rather includes jagged walls, such as walls 400, 401, and 402. Each of the walls 400-402 is slanted at an angle, such that they each have a directional component in both the X-direction and the Y-direction. Collectively, the walls 400-402 define a sawtooth profile. The opening 120 is also defined by a wall 410 extending in the Y-direction and a wall 420 extending in the X-direction. Note that the wall 410 and the wall 420 each have a linear profile in the illustrated embodiment, but this is not required in other embodiments. In other words, the walls 410 and/or 420 may have at least partially curved profiles in other embodiments.

Meanwhile, the hard-stop mechanism 200 in this embodiment comprises a tab with jagged edges, such as edges 500, 501, and 502. Each of the edges 500-502 is also slanted at an angle, such that they each have a directional component in both the X-direction and the Y-direction. The edges 500-502 collectively define a sawtooth profile that coincides with (or is complementary to) the sawtooth profile defined by the walls 400-402. The hard-stop mechanism 200 also has an edge 510 extending in the Y-direction and an edge 520 extending in the X-direction. As will be discussed in more detail below, the hard-stop mechanism 200 will be inserted into the opening 120 when the catheter pull ring 100 is in the ring configuration, and the edges 500-502 of the hard-stop mechanism 200 may come into physical contact with one or more of the walls 400-402 of the opening 120, so as to flexibly configure a size of the catheter pull ring 100. Note that the edge 510 and the edge 520 each have a linear profile in the illustrated embodiment, but this is not required in other embodiments. In other words, the edges 510 and/or 520 may have at least partially curved profiles in other embodiments. The hard-stop mechanism 200 further includes a key hole 530, which allows tweezers or a similar mechanism to extend therethrough, so as to facilitate a better mechanical grip of the hard-stop mechanism 200 when the hard-stop mechanism 200 is inserted into the opening 120.

Note that the wall 410 of the opening has a dimension 550, and the edge 510 of the hard-stop mechanism 200 has a dimension 560. The dimensions 550 and 560 are each measured in the Y-direction, but the dimension 550 is configured to be larger than the dimension 560 according to an aspect of the present disclosure, which will help create a gap between the hard-stop mechanism 200 and the opening 120 when the hard-stop mechanism 200 is inserted into the opening 120. As will be discussed below in more detail, such a gap allows for easier ratcheting of the hard-stop mechanism 200 into the opening 120 to incrementally adjust the size the catheter pull ring 100, which allows a single off-the-shelf device (i.e., the catheter pull ring 100) to be compatible with a wide range of catheter sizes/profiles.

Referring now to FIG. 6, the catheter pull ring 100 is folded or bent into a cylindrical shape (e.g., by a manufacturer of the stainless hypotube in some embodiments), such that the channel 300 discussed above is defined by the folded catheter pull ring 100. In this ring configuration, the hard-stop mechanism 200 is inserted into the opening 120. The jagged walls 400-402 of the opening and the jagged edges 500-502 of the hard-stop mechanism 200 allows the hard-stop mechanism 200 to be ratcheted into different fixed positions within the opening 120, which would lead to different sizes for the channel 300 defined by the catheter pull ring 100. For example, FIG. 6 illustrates the configuration where the channel 300 has a minimum circumference or diameter. In this configuration, the hard-stop mechanism 200 is ratcheted all the way into the opening 120, such that the jagged edges 500-502 of the hard-stop mechanism 200 are in direct physical contact with the jagged walls 400-402 of the opening 120, respectively. Once the hard-stop mechanism 200 is fully inserted into the opening 120, the jagged design (e.g., with the sawtooth profile) herein prevents the hard-stop mechanism 200 from moving further into or out of the opening 120 (e.g., in the X-direction in FIG. 5), which in turn fixes the size of the catheter pull ring 100 (e.g., in terms of the circumference or the diameter of the channel 300).

As discussed above with reference to FIG. 5, the difference between the dimension 550 of the wall 410 of the opening and the dimension 560 of the edge of the hard-stop mechanism 200 results in a gap 580 between the wall 420 of the opening 120 and the edge 520 of the hard-stop mechanism 200. The presence of the gap 580 allows the hard-stop mechanism 200 to be slightly bent in the Y-direction during the ratcheting of the hard-stop mechanism 200 into the opening 120, thereby avoiding contact with the jagged walls 400-402 that could have interfered with the ratcheting process. In addition, the implementation of the key hole 530 in the hard-stop mechanism 200 allows tweezers or a similar mechanism to extend therethrough to provide an enhanced grip of the hard-stop mechanism 200 during the ratcheting process. It is also noted that in the embodiment shown in FIG. 6, the edge 510 of the hard-stop mechanism 200 may make physical contact with the wall 410 of the opening 120. However, this is merely a result of the dimension of the hard-stop mechanism 200 being configured to be substantially equal to the dimension of the opening 120 in the X-direction, which may not necessarily be the case in other embodiment. As such, there may be a slight gap or seam between the edge 510 of the hard-stop mechanism 200 and the wall 410 of the opening 120 in some other embodiments.

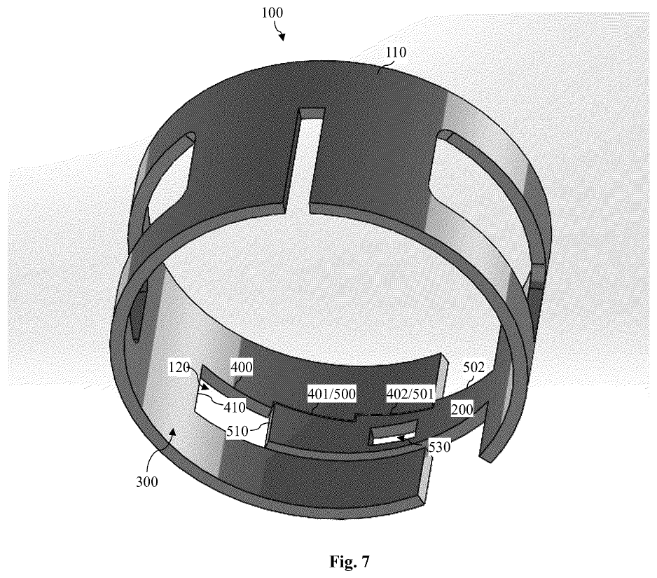

Referring now to FIG. 7, the catheter pull ring 100 is still folded or bent into a cylindrical shape, such that the channel 300 is defined by the folded catheter pull ring 100. However, the channel 300 in the configuration of FIG. 7 achieves a larger circumference or diameter than the configuration of FIG. 6. In that regard, the hard-stop mechanism 200 is partially inserted into the opening 120, but now the edge 500 of the hard-stop mechanism 200 is in direct physical contact with the wall 401 of the opening 120, and the edge 501 of the hard-stop mechanism 200 is in direct physical contact with the wall 402 of the opening 120. The edge 502 of the hard-stop mechanism 200 is no longer in direct physical contact with any part of the body 110, and the wall 400 of the opening 120 is no longer in direct physical contact with any part of the hard-stop mechanism 200. As such, a gap (as a part of the opening 120) exists between the edge 510 of the hard-stop mechanism 200 and the wall 410 of the opening 120. Again, the jagged design (e.g., with the sawtooth profile) of the hard-stop mechanism 200 and the opening 120 prevents the hard-stop mechanism 200 from moving further into or out of the opening 120 (e.g., in the X-direction in FIG. 5), which in turn fixes the size of the catheter pull ring 100, albeit with a larger circumference and diameter than the configuration of FIG. 6. For example, the circumference of the catheter pull ring 100 in the configuration of FIG. 7 is larger than the circumference of the catheter pull ring 100 in the configuration of FIG. 6 by a length of the wall 400 of the opening 120 in the X-direction.

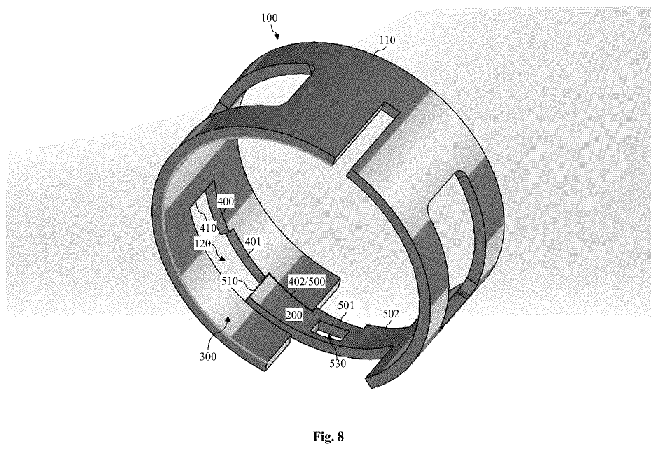

Referring now to FIG. 8, the catheter pull ring 100 is still folded or bent into a cylindrical shape, such that the channel 300 is defined by the folded catheter pull ring 100. However, the channel 300 in the configuration of FIG. 8 achieves an even larger circumference or diameter than the configurations of FIG. 6 and FIG. 7. In that regard, the hard-stop mechanism 200 is partially inserted into the opening 120, but now only the edge 500 of the hard-stop mechanism 200 is in direct physical contact with the wall 402 of the opening 120. The edges 501 and 502 of the hard-stop mechanism 200 are no longer in direct physical contact with any part of the body 110, and the walls 400 and 401 of the opening 120 are no longer in direct physical contact with any part of the hard-stop mechanism 200. As such, an even larger gap (as a part of the opening 120) exists between the edge 510 of the hard-stop mechanism 200 and the wall 410 of the opening 120. Again, the jagged design (e.g., with the sawtooth profile) of the hard-stop mechanism 200 and the opening 120 prevents the hard-stop mechanism 200 from moving further into or out of the opening 120 (e.g., in the X-direction in FIG. 5), which in turn fixes the size of the catheter pull ring 100, albeit with an even larger circumference and diameter than the configurations of FIG. 6 and FIG. 7. For example, the circumference of the catheter pull ring 100 in the configuration of FIG. 8 is larger than the circumference of the catheter pull ring 100 in the configuration of FIG. 7 by a length of the wall 401 of the opening 120 in the X-direction, and larger than the circumference of the catheter pull ring 100 in the configuration of FIG. 6 by a combined length of the walls 400 and wall 401 of the opening 120 in the X-direction.

Note that although FIGS. 6-8 illustrate three different configurations of the catheter pull ring 100 corresponding to three different sizes of the catheter pull ring 100, the same design concepts could be implemented in other embodiments to generate any other number of configurations of the catheter pull ring 100 corresponding to that number of adjustable sizes of the catheter pull ring 100. For example, if four jagged edges/walls (i.e., increasing the number of “teeth” of the sawtooth design from three to four) are implemented for an alternative embodiment, then the catheter pull ring 100 according to that alternative embodiment may be adjusted to four (as opposed to three here) different sizes to accommodate an even greater number of catheter sizes. Furthermore, the geometric design of the jagged edges/walls may also be configured to effectuate different outcomes. For example, changing a height of the teeth of the sawtooth design (i.e., changing the Y-direction component of the walls 400-402 and edges 500-501) may translate into a different amount of mechanical interference between the hard-stop mechanism 200 and the opening 120 to lock the hard-stop mechanism 200 in a fixed position. The greater the height of the teeth of the sawtooth design, the easier it is to lock the hard-stop mechanism 200 in place, but the hard-stop mechanism 200 may need to be bent farther (in the Y-direction) as it is ratcheted in the opening 120. As another example, changing the length of the hard-stop mechanism 200 and the corresponding opening 120 in the X-direction would affect the range of circumferences or diameters that can be created with a single catheter pull ring 100 (as a cylindrical tube).

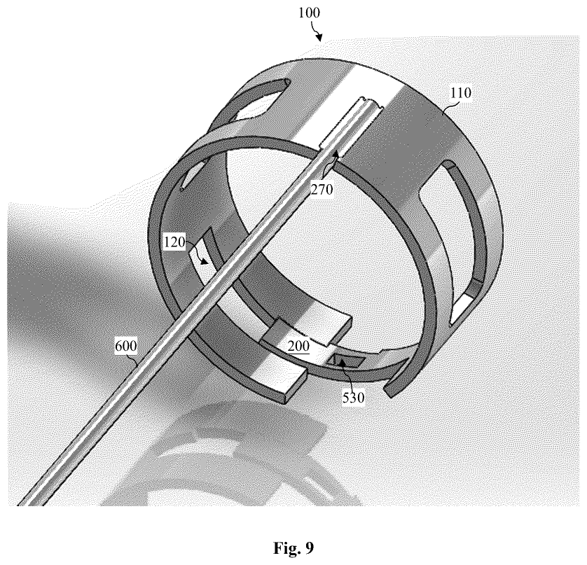

Referring now to FIG. 9, another three-dimensional perspective view of the catheter pull ring 100 is illustrated. However, the view in FIG. 9 illustrates not just the catheter pull ring 100, but also an attachment mechanism 600 that is coupled to the catheter pull ring 100. As discussed above, one of the functions of the catheter pull ring 100 is to facilitate a deflection of the catheter in one or more given directions. Such a directional deflection may be achieved by pulling on the attachment mechanism 600, which in this embodiment includes a metal wire that is inserted into the slot 270. The attachment mechanism 600 wire may be laser welded (or otherwise affixed) to the catheter pull ring 100. A handle assembly located at a proximal end of the catheter may be actuated to pull the attachment mechanism 600 in the proximal direction, which in turn will cause the catheter pull ring 100 to deflect in a direction toward the proximal end of the catheter. Note that although just one attachment mechanism 600 is illustrated herein, this is done for reasons of simplicity. In other embodiments, any other number (e.g., two or more) attachment mechanisms may be attached to other locations of the catheter pull ring 100, so as to allow the catheter pull ring 100 to be deflected in other directions by actuating the handle assembly.

Referring now to FIG. 10, a three-dimensional perspective view of a catheter assembly 700 is illustrated. The catheter assembly 700 includes the catheter pull ring 100, the attachment mechanism 600 attached to the catheter pull ring 100, as well as a catheter shaft 710. As discussed above, a distal end of the catheter shaft 710 may be inserted into a channel (e.g., the channel 300 discussed above) defined by the catheter pull ring 100, which is then attached to the catheter shaft 710. The catheter pull ring 100 serves as an anchor or a stabilization mechanism for the catheter shaft 710 by securing the catheter shaft 710 in place and ensuring that it remains properly positioned during use. The catheter pull ring 100 also provides structural support for the catheter shaft 710 by providing rigidity or a point of attachment for other components, such as for the attachment mechanism 600, so as to aid in the proper deployment and functionality of the catheter assembly 700 (e.g., facilitating directional deflection of the catheter shaft 710). In addition, the catheter pull ring 100 may serve as radiopaque markers to allow visualization under X-rays or other imaging techniques, thereby helping healthcare providers confirm the position of the catheter assembly 700.

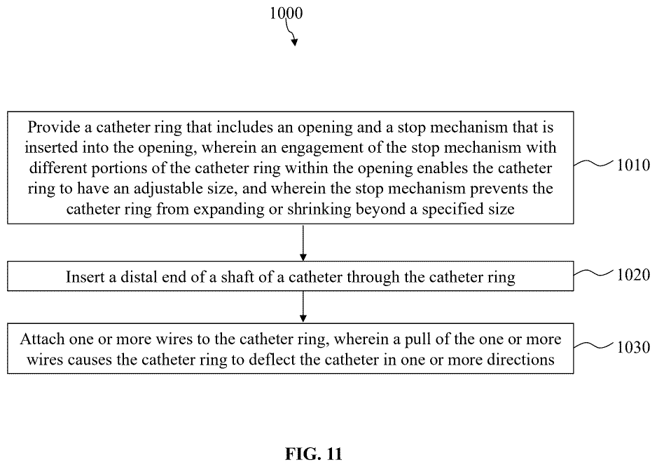

Referring now to FIG. 11, a flowchart of a method 1000 of the present disclosure is illustrated. The method 1000 includes a step 1010, in which a catheter pull ring is provided. The catheter pull ring may be the catheter pull ring 100 discussed above in association with FIGS. 1-10. The catheter pull ring includes an opening and a stop mechanism that is inserted into the opening. For example, the opening may be the opening 120 discussed above, and the stop mechanism may be the hard-stop mechanism 200 discussed above. An engagement of the stop mechanism with different portions of the catheter pull ring within the opening enables the catheter pull ring to have an adjustable size (e.g., in terms of circumference or diameter). The stop mechanism prevents the catheter pull ring from expanding or shrinking beyond a specified size (e.g., from expanding beyond a maximum circumference/diameter or shrinking beyond a minimum circumference/diameter). In some embodiments, the stop mechanism may include a T-shaped bar, such as the hard-stop mechanism 200 illustrated in FIGS. 1-4. In some other embodiments, the stop mechanism may include a tab with jagged edges (e.g., defining a sawtooth profile), such as the hard-stop mechanism 200 illustrated in FIGS. 5-8.

The method 1000 includes a step 1020, in which a distal end of a shaft of a catheter is inserted through the catheter pull ring. For example, a distal end of the catheter shaft 710 may be inserted through a channel defined by the catheter pull ring, as discussed above with reference to FIG. 10. Also as discussed above, the catheter pull ring may be attached to the catheter shaft to provide mechanical rigidity and to serve as an anchoring mechanism for the catheter shaft.

The method 1000 includes a step 1030, in which one or more wires are attached to the catheter pull ring. For example, the one or more wires may include the attachment mechanism 600 discussed above. A pull of the one or more wires (e.g., via an actuation of a handle mechanism located at a proximal end of the catheter) causes the catheter pull ring to deflect the catheter in one or more directions.

Note that the steps 1020 and 1030 need not be performed sequentially. In some embodiments, the step 1030 may be performed before the step 1020. In other words, the one or more wires may be attached to the catheter pull ring before the distal end of the catheter shaft is inserted into, and attached to, the catheter pull ring. It is also understood that the method 1000 may include additional steps that are performed before, during, or after the steps 1010, 1020, and/or 1030. For example, the method 1000 may include a step of manufacturing the catheter pull ring, a step of bending the catheter pull ring from a flat configuration into the ring configuration, a step of ratcheting the hard stop mechanism into the opening, a step of placing the catheter in a human body, and/or a step of causing a directional deflection of the distal end of the catheter via an actuation of a handle assembly, etc. These steps are not discussed in detail herein for reasons of simplicity.

The embodiments of the catheter pull rings of present disclosure offer advantages over conventional catheter pull rings. It is understood, however, that other embodiments may offer additional advantages, and not all advantages are necessarily disclosed herein, and that no particular advantage is required for all embodiments. One advantage is the improved customizability with respect to the size of the catheter pull ring. Conventional catheter pull rings (particularly as off-the-shelf devices) typically come in at fixed sizes. As such, there is a very limited range of catheters that may be compatible with any given conventional catheter pull ring. If the size of the catheter (e.g., in terms of circumference or diameter) is too small relative to the size of the catheter pull ring, it may be difficult to properly couple the catheter pull ring and the catheter together. On the other hand, if the size of the catheter is too large relative to the size of the catheter pull ring, the catheter pull ring may not be able to accommodate the catheter. In contrast, the catheter pull ring of the present disclosure has an adjustable size. For example, by moving the hard-stop mechanism 200 to different positions within the opening 120, the catheter pull ring can achieve different circumferences/diameters. The design of the hard-stop mechanism 200 and the opening 120 also allows the hard-stop mechanism 200 to stay at a fixed location, which prevents the catheter pull ring from shrinking or expanding any further, thereby affixing a particular desired size for the catheter pull ring via mechanical design. The sizing flexibility for the catheter pull ring herein makes the catheter pull ring particularly attractive as an off-the-shelf device, since it may accommodate a variety of catheter sizes without having to be custom-made, which may significantly shorten lead time. In addition, the implementation of the key hole 530 in the hard-stop mechanism 200 (see FIGS. 5-8) allows tweezers or other mechanical tools to provide a better grip of the hard-stop mechanism 200 as the hard-stop mechanism is ratcheted through the opening 120. Furthermore, the implementation of the gap 580 (see FIG. 6) between the hard-stop mechanism 200 and the opening 120 allows the hard-stop mechanism 200 to be slightly bent and therefore ratcheted more easily through the opening 120. Other advantages include compatibility with existing manufacturing processes and the ease and low cost of implementation.

One aspect of the present disclosure pertains to an apparatus. The apparatus includes a body of a catheter pull ring that is folded into a cylindrical shape. The apparatus includes an opening in the body. The apparatus includes a hard-stop mechanism of the catheter pull ring that is configured to be inserted into the opening when the body is folded into the cylindrical shape. The hard-stop mechanism is configured to engage with a portion of the body around the opening, such that the body that has been folded into the cylindrical shape is prevented from expanding or shrinking a circumference thereof beyond a predefined threshold.

Another aspect of the present disclosure pertains to an apparatus. The apparatus includes a body configured to be bent into a ring that is fittable within a shaft of a catheter. The apparatus includes an opening in the body. The opening is located at a first end portion of the body. The apparatus includes a stop mechanism extending from a second end portion of the body opposite the first end portion. When the body is bent into the ring, the stop mechanism is inserted into the opening and is configured to engage with different portions of the body. Engagements of the stop mechanism with the different portions of the body correspond to different sizes of the ring.

Yet another aspect of the present disclosure pertains to a method. A catheter pull ring is provided. The catheter pull ring includes an opening and a stop mechanism that is inserted into the opening. An engagement of the stop mechanism with different portions of the catheter pull ring within the opening enables the catheter pull ring to have an adjustable size. The stop mechanism prevents the catheter pull ring from expanding or shrinking beyond a specified size. A distal end of a shaft of a catheter is inserted through the catheter pull ring. One or more wires are attached to the catheter pull ring. A pull of the one or more wires causes the catheter pull ring to deflect the catheter in one or more directions.

The foregoing outlines features of several embodiments so that those of ordinary skill in the art may better understand the aspects of the present disclosure. Those of ordinary skill in the art should appreciate that they may readily use the present disclosure as a basis for designing or modifying other processes and structures for carrying out the same purposes and/or achieving the same advantages of the embodiments introduced herein. Those of ordinary skill in the art should also realize that such equivalent constructions do not depart from the spirit and scope of the present disclosure, and that they may make various changes, substitutions, and alterations herein without departing from the spirit and scope of the present disclosure.

What is claimed is:

1. An apparatus, comprising:
a body of a catheter pull ring that is folded into a cylindrical shape;
an opening in the body; and
a hard-stop mechanism of the catheter pull ring that is configured to be inserted into the opening when the body is folded into the cylindrical shape, wherein the hard-stop mechanism is configured to engage with a portion of the body around the opening, such that the body that has been folded into the cylindrical shape is prevented from expanding or shrinking a circumference thereof beyond a predefined threshold.

2. The apparatus of claim 1, further comprising a catheter shaft, wherein the body of the catheter pull ring, once folded into the cylindrical shape, is configured to be fitted within a distal end of the catheter shaft.

3. The apparatus of claim 2, further comprising one or more wires attached to the body of the catheter pull ring, wherein a pull of the one or more wires causes the catheter shaft to be deflected in one or more directions.

4. The apparatus of claim 1, wherein the hard-stop mechanism comprises a T-shaped bar.

5. The apparatus of claim 4, wherein:
the body extends in a first direction;
the portion of the body comprises one or more protruding segments that each protrude in a second direction different from the first direction;
one or more first walls of the opening are defined by the one or more protruding segments;
a second wall of the opening is defined by a further portion of the body;
the second wall extends in the second direction and faces against the one or more first walls; and
the predefined threshold occurs when the T-shaped bar is in physical contact with the one or more first walls or with the second wall.

6. The apparatus of claim 5, wherein the circumference of the folded cylindrical body is at a maximum value when the T-shaped bar is in physical contact with the one or more first walls.

7. The apparatus of claim 5, wherein the circumference of the folded cylindrical body is at a minimum value when the T-shaped bar is in physical contact with the second wall.

8. The apparatus of claim 1, wherein the hard-stop mechanism comprises a tab with a jagged edge.

9. The apparatus of claim 8, wherein:
the jagged edge of the tab has a first jagged profile;
a first wall of the opening has a second jagged profile that is complementary to the first jagged profile; and
the jagged edge of the tab is in physical contact with the first wall of the opening when the body is folded into the cylindrical shape.

10. The apparatus of claim 9, wherein when the jagged edge of the tab is in physical contact with different portions of the first wall of the opening, a size of the folded cylindrical body reaches different values.

11. The apparatus of claim 9, wherein:
the jagged edge is a first edge of the tab; and
a second edge of the tab opposite the first edge and a second wall of the opening opposite the first wall each have a linear profile.

12. The apparatus of claim 11, wherein the second edge and the second wall are separated by a gap.

13. The apparatus of claim 8, wherein the tab includes a key hole.

14. An apparatus, comprising:
a body configured to be bent into a ring that is fittable within a shaft of a catheter;
an opening in the body, wherein the opening is located at a first end portion of the body; and
a stop mechanism extending from a second end portion of the body opposite the first end portion, wherein when the body is bent into the ring, the stop mechanism is inserted into the opening and is configured to engage with different portions of the body, and wherein engagements of the stop mechanism with the different portions of the body correspond to different sizes of the ring.

15. The apparatus of claim 14, wherein:
the body extends in a first direction;
the stop mechanism comprises a T-shaped tab;
the first end portion of the body includes one or more protruding segments that each protrude in a second direction different from the first direction;
the opening is partially defined by the one or more protruding segments; and
the ring reaches a maximum size when the stop mechanism is in physical contact with the one or more protruding segments.

16. The apparatus of claim 14, wherein:
the stop mechanism comprises a tab that includes an edge having a sawtooth profile;
a wall of the opening also has the sawtooth profile; and
the edge of the tab makes physical contact with the wall of the opening when the body is bent into the ring.

17. The apparatus of claim 16, wherein:
the edge having the sawtooth profile is a first edge of the tab;
the wall of the opening having the sawtooth profile is a first wall of the opening;
the tab further includes a second edge opposite the first edge;
the opening further includes a second wall opposite the first wall; and
the second edge and the second wall each have a linear profile.

18. The apparatus of claim 17, wherein the second edge and the second wall are spaced apart by a gap.

19. A method, comprising:
providing a catheter pull ring that includes an opening and a stop mechanism that is inserted into the opening, wherein an engagement of the stop mechanism with different portions of the catheter pull ring within the opening enables the catheter pull ring to have an adjustable size, and wherein the stop mechanism prevents the catheter pull ring from expanding or shrinking beyond a specified size;
inserting a distal end of a shaft of a catheter through the catheter pull ring; and
attaching one or more wires to the catheter pull ring, wherein a pull of the one or more wires causes the catheter pull ring to deflect the catheter in one or more directions.

20. The method of claim 19, wherein the stop mechanism comprises a T-shaped bar or a tab with a jagged edge.
